(19) United States
(12) Patent Application Publication
(10) Pub. No.: US 2007/0162116 A1
(43) Pub. Date: Jul. 12, 2007

(54) Method for locating optimum presbyopia implant location

(76) Inventor: Georges Baikoff, Marseille (FR)

Correspondence Address: SHELDON MAK ROSE & ANDERSON PC, 100 East Corson Street, Third Floor, PASADENA, CA, 91103-3842, US

(21) Appl. No.: 11/330,678

(22) Filed: Jan. 11, 2006

(51) Int. Cl. A61F 2/14

(52) U.S. Cl. 623/4.1; 623/905

(57) ABSTRACT

A method for implanting a corrective element into the sclera of an eye for the treatment of presbyopia comprises the steps of (a) identifying an optimum implantation location by pressing against different alternative locations along the sclera until a marked shifting of the equator of the crystalline lens is observed, and (b) implanting a corrective element within the sclera at the optimum location.

### BACKGROUND OF THE INVENTION

Presbyopia is a loss or reduction of the accompanying power of the eye which takes place when a person ages. FIG. 1 is a diagrammatic representation of an eye 1 having a cornea 14, showing the lens 2 enclosed in the lens sac 3 and suspended from the ciliary body 4 by means of the zonule 5. The ciliary body 4 lines the internal surface of the sclera 6 about a ring located on the average at a latitude distance by 2 to 3 mm from the limbus 7, measured along the optical axis.

New methods for treating presbyopia have recently been disclosed wherein implant elements are disposed within small tunnels formed within the sclera of the patient's eyes. Once disposed in the scleral tunnels, the implants act on the sclera to enhance the ability of the patient's lens to contract, thereby diminishing the presbyopia condition. Examples of such new methods are disclosed, for example, in my previously-issued patents, U.S. Pat. Nos. 6,682,560 and 6,692,524, the entireties of which are incorporated herein by this reference.

FIG. 2 illustrates one of these new methods. An implant element 8 is surgically disposed within an incision tunnel 9 formed in the sclera 6 opposite the zonule 5. The tunnel 9 is disposed at a depth of about 600μ below the surface of the sclera 6. The tunnel 9 is typically about 8 mm long and about 3 mm wide. The implant element 8 favorably effects the adjustment of the lens shape by the sclera 6 and the zonule 5 to minimize the effects of presbyopia.

In such new methods, it has not been clear as to where to optimally locate the implant elements in the sclera. Accordingly, there is a need for a method for implanting implant elements into the sclera of a presbyopia patient whereby the insert elements are disposed at optimum locations.

### SUMMARY OF THE INVENTION

The invention satisfies this need. The invention is a method of implanting a corrective element into the sclera of an eye for the treatment of presbyopia, the method comprising the steps of (a) identifying an optimum implantation location by pressing against different alternative locations along the sclera until a marked shifting of the equator of the crystalline lens is observed, and (b) implanting a corrective element within the sclera at the optimum location.

### DRAWINGS

These and other features, aspects and advantages of the present invention will become better understood with reference to the following description, appended claims and accompanying drawings where:

FIG. 1 is a schematic view in cross-section of an eye;

FIG. 2 is a fragmentary cross-sectional view of an eye in which is implanted a corrective element for the treatment of presbyopia;

FIG. 3 is a diagrammatic front view of an initial phase of a first step in the method of the invention;

FIG. 4 is a fragmentary cross-sectional view of an eye illustrating the initial phase of the first step in the method of the invention;

FIG. 5 is a diagrammatic front view of a later phase in the first step of the method of the invention;

FIG. 6 is a diagrammatic front view of a still later phase in the first step of the method of the invention; and

FIG. 7 is a fragmentary cross-sectional side view of an eye illustrating the still later phase in the method of the invention.

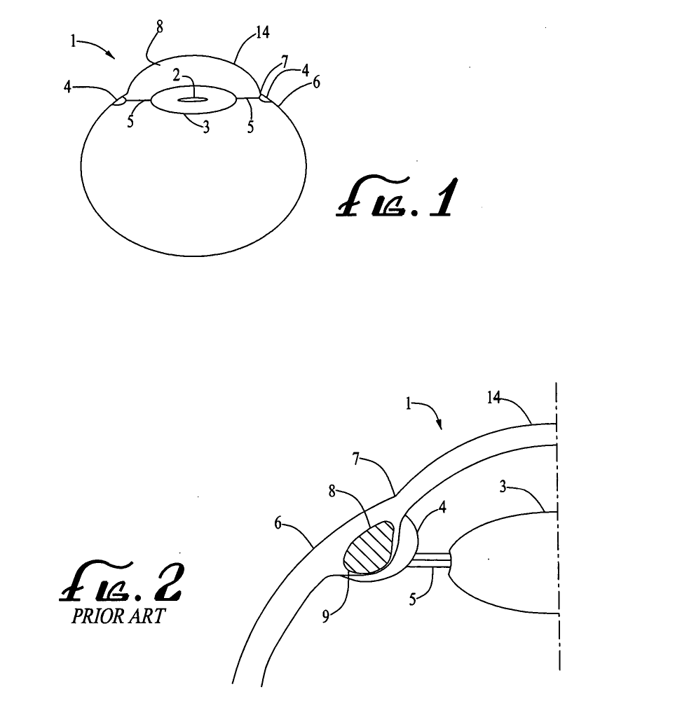

### DETAILED DESCRIPTION

The following discussion describes in detail one embodiment of the invention and several variations of that embodiment. This discussion should not be construed, however, as limiting the invention to those particular embodiments. Practitioners skilled in the art will recognize numerous other embodiments as well.

The invention is a method of implanting a corrective element 8 into the sclera 6 of a eye 1 for the treatment of presbyopia.

In a first step of the method, an optimum implantation location is identified by pressing against different alternative locations along the sclera 6 until marked shifting of the equator of the crystalline lens 2 is observed. Typically, the pupil is dilated first. Then the sclera 6 is progressively pressed down, such as with a forward/backward movement. Such progressive pressing down against the sclera 6 can be conveniently begun at the limbus 7. The optimum implantation location can thereupon be marked with a diathermy.

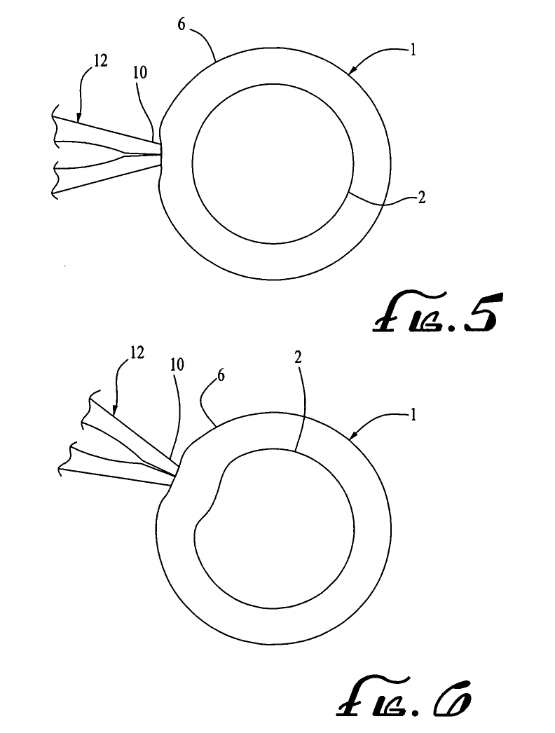

Such pressing down on the sclera 6 can be achieved with the forward tip 10 of a forceps instrument 12, as illustrated in the drawings. FIGS. 3 and 4 illustrate the forward tip 10 of a forceps instrument 12 approaching the sclera 6 of the eye 1 of a patient. FIG. 5 illustrates the pressing against different alternative locations along the sclera 6 with the forward tip 10 of the forceps instrument 12.

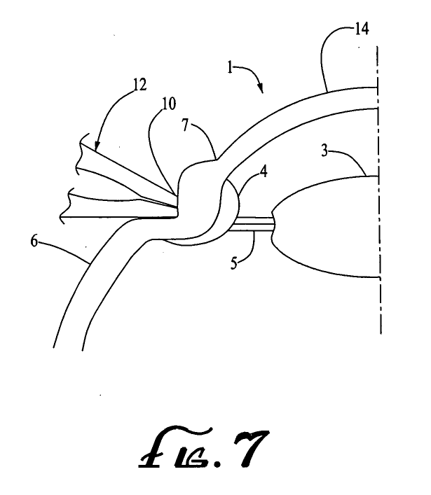

FIGS. 6 and 7 illustrate what happens when scleral force is applied at an optimum implantation location. The scleral force pushes inwardly and substantially coaxially with the zonule 5, causing a shortening of the distance between the sclera 6 and the lens sack 3. This shortening of the distance between the sclera 6 and the lens sack 3 results in a relaxation of the zonule 5 opposite where the sclera 6 is being pressed, and is visible by a corresponding change in the shape of the crystalline lens equator.

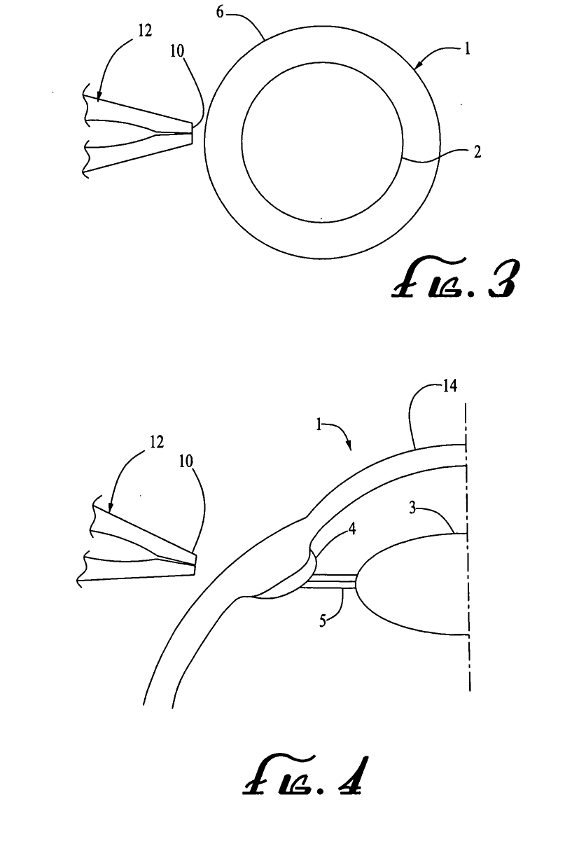

After the optimum implantation location within the sclera 6 is identified, in a second step in the method of the invention, a corrective element 8 is implanted at the optimum implantation location, as illustrated in FIG. 2.

Having thus described the invention, it should be apparent that numerous structural modifications and adaptations may be resorted to without departing from the scope and fair meaning of the instant invention as set forth hereinabove and as described hereinbelow by the claims.

What is claimed is:

1. A method for implanting a corrective element into the sclera of an eye for the treatment of presbyopia, the eye further comprising a crystalline lens and zonule, the method comprising the steps of:
(a) identifying an optimum implantation location by pressing against different alternative locations along the sclera until a marked shifting of the equator of the crystalline lens is observed; and
(b) implanting a corrective element within the sclera at the optimum location.

2. The method of claim 1 wherein, prior to the identifying of an optimum implantation location in step (a), the pupil of the eye is dilated.

3. The method of claim 1 wherein, after the identifying of an optimum implantation location in step (a), the identified optimum implantation location is marked.

4. The method of claim 3 wherein the marking of the optimum implantation location is accomplished with a diathermy.

5. The method of claim 1 wherein the pressing against different alternative locations along the sclera is accomplished using a forceps instrument.
